(19) United States
(12) Patent Application Publication
(10) Pub. No.: US 2007/0100216 A1
(43) Pub. Date: May 3, 2007

(54) Psycho/physiological deception detection system and method for controlled substance surveillance

(76) Inventors: Mark T. Radcliffe, Shady Spring, WV (US); Angela L. Radcliffe, Shady Spring, WV (US)

Correspondence Address: LITMAN LAW OFFICES, LTD., P.O. BOX 15035, CRYSTAL CITY STATION, ARLINGTON, VA, 22215, US

(21) Appl. No.: 11/515,908

(22) Filed: Sep. 6, 2006

Related U.S. Application Data
(60) Provisional application No. 60/731,878, filed on Nov. 1, 2005.

(51) Int. Cl. A61B 5/00; G06F 19/00; A61B 19/00

(52) U.S. Cl. 600/300; 705/3; 600/557

(57) ABSTRACT

The psycho/physiological deception detection (PDDS) is a system and method employing PDDS tools to ensure patient truthfulness while conducting a patient interview to assess the severity of pain suffered by the patient and to assess the risk that the patient will divert controlled substances prescribed for the patient to an illicit use. The patient is monitored using the PDDS tool while making an oral response (either live or off-site) to each question in the interview, and a response and a measure of truthfulness of the patient generated from the PDDS monitoring is correlated with each question. Deceptive responses may be probed by asking refining questions to explore possible innocent explanation for a deceptive response. Based on the patient's responses and the indication of truthfulness for each response, a report is generated from which the patient's risk for diverting prescribed medication and the patient's severity of pain may be assessed.

### CROSS-REFERENCE TO RELATED APPLICATION

This application claims the benefit of U.S. Provisional Patent Application Ser. No. 60/731,878, filed Nov. 1, 2005.

### BACKGROUND OF THE INVENTION

1. Field of the Invention

The present invention relates to systems and methods for administering prescribed medications, and more particularly to systems and methods of surveillance for controlled substance medications.

2. Description of the Related Art

A significant number of people suffer from severe chronic or acute pain. The pain may be the result of disease, injury, trauma caused by surgical procedures, conditions caused by aging, or from other causes. For patients suffering the most severe and persistent pain, the most effective treatment strategies often involve the use of controlled substance medications, such as opioids.

While use of controlled substance medications provides effective treatment when used in appropriate treatment regimens, controlled substance medications also pose a risk of misuse. Some controlled substance medications may pose the risk of physical or psychological dependencies or addiction when taken inappropriately. Further, a significant number of people enjoy taking some controlled substances to experience psychotropic, euphoric, or other side effects that are deemed as pleasurable or recreational.

The desire of a large number of people to exploit controlled substances for illicit purposes presents a lucrative market for the sale of controlled substances for non-prescribed and illegal uses.

In one scheme for regulating controlled substance medications, the medications are classified into schedules. Schedule I substances are those substances with a high potential for abuse, and which have no accepted medical or therapeutic value. Schedule II medications are those with accepted medical value, but which also have a substantial potential for abuse or dependency. Medications classified in the respective Schedules III, IV and V have successively lesser risks for abuse and have accepted therapeutic uses. Schedule V medications possess only a small potential for abuse or risk of dependency. The controlled substances classified in Schedule I are illegal to use or prescribe, while those in Schedule V are subject to relatively light regulation and may be available without a prescription.

However, medications in Schedules II, III, and IV are subject to significant government controls, with Schedule II medications being subject to the most restrictive regulations. These regulations place responsibilities on prescribing physicians to verify that prescribed controlled medications are properly administered and used by patients. Physicians who suspect patterns of abuse by a patient have moral and often legal responsibilities to intervene to arrest the abuse. Medications in Schedules II, III, and IV all require prescriptions from a physician. Schedule II may be prescribed in limited amounts with prescriptions not to be refilled, while medications in Schedule III and IV may be refilled a controlled number of times over a limited period without revisiting a physician.

Physicians wishing to insure the proper use of medications may undertake a number of surveillance actions to ensure that the medications are not abused. For example, a prescribing physician may require that a patient agree to pill counts. In a pill count, the patient demonstrates compliance with the prescribed use of a controlled substance medication by presenting the unused portion of the prescription for counting by the physician. Counting limits the possibility that the patient is supplying prescribed medications to others. Physicians may also attempt to qualify patients by subjecting them to drug testing or screening procedures, such as urinalysis. Physicians may disqualify patients identified as drug abusers as the result of drug screening as candidates for receiving prescriptions of controlled substance medications. The use of these procedures to qualify patients adds to the expense and administrative overhead associated with prescribing medications to deserving patients. Additionally, methods such as drug screening and pill counts are not foolproof. Methods for defeating many types of drug screening tests are well known. Patients may defeat pill count surveillance by obtaining prescriptions from multiple physicians and presenting each physician with the expected amount of remaining prescription, even while diverting pills for illicit use by themselves or others.

The described surveillance strategies may be ineffective against people who attempt to simulate pain symptoms in order to deceive the physician into prescribing a controlled substance medication. Since the deceiving person is often not actually suffering from pain and has no need to take the prescribed medication, the entire prescribed amount of the medication may be diverted at the prescribed dosage rate to illicit use without the diversion being detectable by such methods as pill counts. If the false patient is diverting the medication for distribution to others, drug screening will not detect the pattern of drug abuse.

For patients with no history or inclination towards drug abuse, controlled substance medications, including those in Schedule II, may be the safest and most effective treatment options. Physicians who are unsure of the risks associated with a specific patient, however, may avoid prescribing a controlled substance medication, or may prescribe a controlled substance medication with a lower potential for abuse, such as a Schedule IV or Schedule V medication. These medications may be less effective for the treatment of pain, reducing the quality of life for a patient despite the patient's personal low risk for abuse of an effective medication. The substituted medications may also have side effects that have adverse effects on the health or quality of life of the patient, even when the substitutes are effective at treating the patient's pain. Alternatively the physician may implement a costly surveillance system for patients for which the risk of abuse is low.

Regulatory agencies observing patterns of abuse of Schedule III, IV, and V controlled substance medications may elect to reclassify an abused medication into a more restrictive schedule, such as Schedule II. While this reclassifying action does result in a reduced incidence of abuse of the rescheduled controlled substance due to the reduction in prescriptions written for patients, the reduction has the undesirable effect of reducing access to medications by patients who would safely use the medications.

If physicians were able to identify patients in sincere need of a controlled substance medication for treating an actual acute or chronic severe pain condition, and who additionally presented a minimal risk for abusing the medications, such as opioids, for recreation use, doctors could prescribe the most effective available treatments for those safe patients. The lessened patterns of abuse would result in less motivation to further restrict legitimate access to controlled substance medications. The knowledge that doctors are effectively able to detect fraudulent attempts to obtain controlled substance medications would deter persons from making deceptive attempts to procure the controlled substances. Ideally, the apparatus or technique would be more reliable and less intrusive than present surveillance methods.

French Patent No. 2,624,373, published Jun. 16, 1989, and French Patent No. 2,801,186, published May 25, 2001, describe devices for quantifying the pain experienced by a person. These documents are relevant to show the state of the art with respect to assessment of pain, but do not describe inventions performing the same function or with the same structure as the instant invention.

Thus, a psycho/physiological deception detection system and method for controlled substance surveillance solving the aforementioned problems is desired.

### SUMMARY OF THE INVENTION

The psycho/physiological deception detection (PDDS) system and method employs PDDS tools to determine patient truthfulness while conducting a patient interview to assess the severity of pain suffered by the patient and to assess the risk that the patient will divert a controlled substance prescribed for the patient to an illicit use. The patient is prepared as required for employment of the particular PDDS tool chosen, including connecting required PDDS sensors to the patient and obtaining baseline data correlated with truthful and untruthful responses by the patient.

The patient is monitored (either live or off-site) using the PDDS tool while making an oral response to each question in the interview, and a response and a measure of truthfulness of the patient generated from the PDDS monitoring is correlated with each question. Deceptive responses may be probed by asking refining questions to explore possible innocent explanation for a deceptive response. Based on the patient's responses and the indication of truthfulness for each response, a report is generated from which the patient's risk for diverting prescribed medication and the patient's severity of pain may be assessed. Biometric data, such as facial images, are collected from the patient during the interview and may be used to detect repeated attempts to obtain controlled substance medications.

The PDDS tool may employ any technology capable of detecting indications of stress and correlating the indications of stress with an attempt at deception while making an oral response. The PDDS tool may be a polygraph; voice stress analysis tools, such as Layered Voice Analysis (LVA) or Computer Voice Stress Analysis (CVSA); a facial gesture analysis tool, such as Silent Talker; or any other tool for monitoring patient stress while providing an oral response. The patient's oral response may also be recorded and sent for veracity analysis off-site in another location.

The system includes a computer readable medium for storing data in data structures. The data structures contain records containing fields that are operable to control devices, such as a computer, to implement aspects of the psycho/physiological deception detection (PDDS) method. The computer readable medium may be storage memory on a server accessible over a network. The records are made up of information fields. A first information field holds patient identification data for identifying records associated with a specified patient. Optionally, the identification field holds biometric data. Each record further includes a patient interview field operable to specify contents of a patient assessment report. A record may further include a patient release field operable to specify persons authorized to receive the patient assessment report.

These and other features of the present invention will become readily apparent upon further review of the following specification and drawings.

### BRIEF DESCRIPTION OF THE DRAWINGS

FIG. 1 is a block diagram of a networked psycho/physiological deception detection system according to the present invention.

FIG. 2 is a block diagram of an exemplary computer on which the system and method of the present invention may be implemented.

FIG. 3 is a flowchart of a psycho/physiological deception detection process for assessing a patient's suitability to receive a prescription for a controlled substance according to the system and method of the present invention

FIG. 4 is a flowchart of a psycho/physiological deception detection process for assessing the severity level of pain suffered by a patient according to the system and method of the present invention.

FIG. 5 is a flowchart of a psycho/physiological deception detection process for assessing the risk of diversion of a controlled substance associated with a patient according to the system and method of the present invention.

FIG. 6 is a flowchart of a process for generating a patient assessment report according to the system and method of the present invention.

FIG. 7 is a block diagram of a patient interview data structure according to the system and method of the present invention.

FIG. 8 is a block diagram of an interview history data structure according to the system and method of the present invention.

FIG. 9 is a depiction of a patient pain assessment response report according to the system and method of the present invention.

FIG. 10 is a depiction of a patient diversion risk assessment response report according to the system and method of the present invention.

Similar reference characters denote corresponding features consistently throughout the attached drawings.

### DETAILED DESCRIPTION OF THE PREFERRED EMBODIMENTS

The present invention is a system and method for assessing the suitability of a patient to receive a controlled substance medication for the purpose of treating pain suffered by the patient by conducting a patient interview, assessing the patients responses, either live during the interview or off-site, including detecting and assessing attempts by the patient to provide deceptive responses during the interview and correlating the interview information with other relevant information. The method includes the employment of psycho/physiological deception detection surveillance (PDDS) tools to detect deceptive responses. As part of the method, an assessment report is generated for use in making a decision to prescribe or not to prescribe controlled substance medications to the patient.

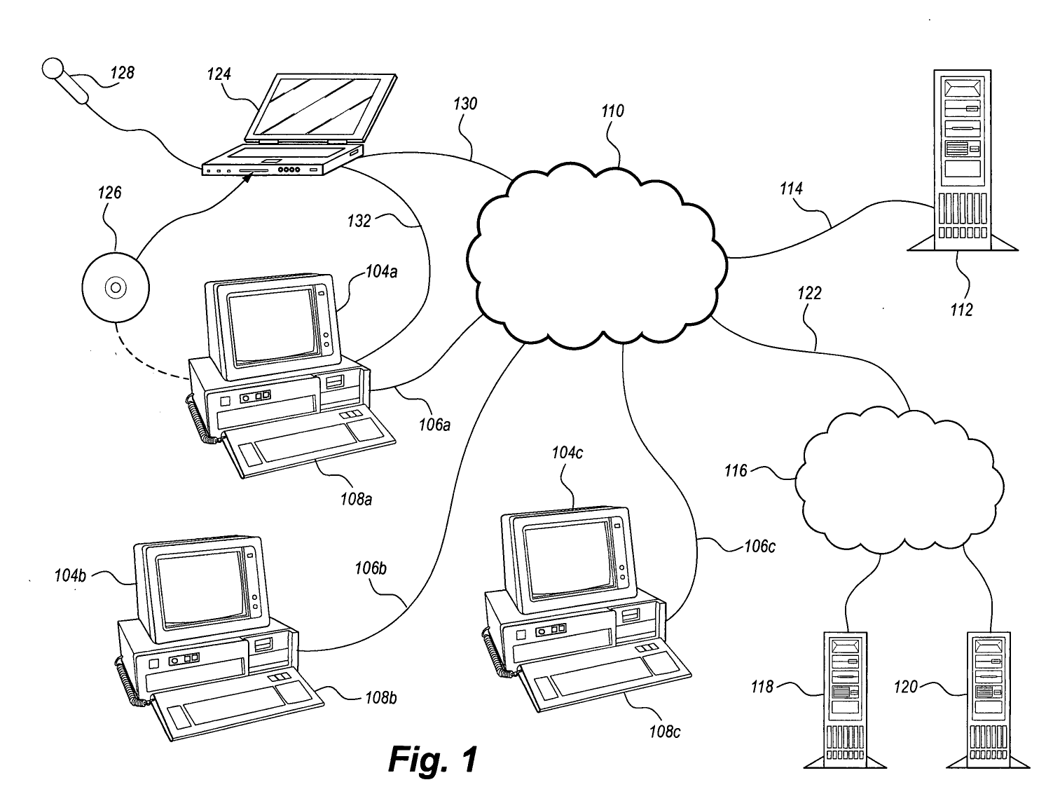

An embodiment of an exemplary system for assessing the suitability of a patient to receive a controlled substance medication is illustrated in FIG. 1. The system includes a computerized interview workstation 124. The interview workstation 124 may be a portable computer, such as a laptop or notebook computer. Software on the interview workstation 124 facilitates the conduct of patient interviews in accordance with the invention by receiving personal identification information associated with the patient, retrieving and displaying the interview questions, and collecting information related to detection of deception by an interviewed patient responding to the interview questions. A microphone 128 is connected to the interview workstation 124. A patient's responses to interview questions may be captured and transferred to the interview workstation 124 via the microphone 128 and analyzed using software stored on the interview workstation 124 to detect attempts by the patient to provide deceptive responses during an interview.

Information gathered during the interview process may be transferred to a centralized database for storage and retrieval. A database server 112 is attached to a first network 110. This network 110 may be a wide area network (WAN), an intranet or extranet, or the Internet. The database server 112 is connected to the network 110 via a connecting link 114. One or more central station computers 104a, 104b, and 104c are connected by networking links 106a, 106b, and 106c to the first network 110. These networking links may be wired or wireless connections, and are preferably high-speed connections to the network 110. While three central station computers are shown, any number of central station computers may be employed.

Data may be transferred from the interview workstation 124 to the central station network 110 over a network link 130, and then transferred over the network to one of the central station computers, such as computer 104a, or to the central database 112. Alternatively, data may be transferred to one of the central station computers, such as computer 104a, by writing the data to a removable computer readable medium, such as a CD, DVD or other optical disk 126, and physically transferring the disk to an appropriate reader connected to one of the networked central station computers 104a, 104b, or 104c. Data collected during an interview may be manually entered into one of the networked central station computers via a keyboard 108a, 108b, or 108c attached to the respective central station computer 104a, 104b, or 104c.

Data may alternatively be transferred between the interview workstation 124 and a central station computer via a computer-to-computer link 132 between the computers. The computer-to-computer link 132 may be a wired connection, such as a computer link employing the Universal Serial Bus (USB) protocol, or the connection may be a wireless connection, such as a radio frequency (RF) link employing the Bluetooth protocol.

The central station network may be connected to one or more external networks 116 via a network-to-network link 122 or by a connection over the Internet. The connection to an external network allows the transfer of data to and from remote database servers 118 and 120. The facilities for connection to remote database servers provides access to externally compiled information that may be used to assess a patient's suitability for receiving prescriptions of controlled substance medications. For example, the database servers 118 or 120 may store criminal record information, law enforcement reports, or other relevant information.

As may be appreciated by one skilled in the art, the described methods for transferring data between computers and servers on the network 110 and the interview workstation 124 are only examples, and other methods of transferring data between computers may be employed without departing from the spirit of the invention.

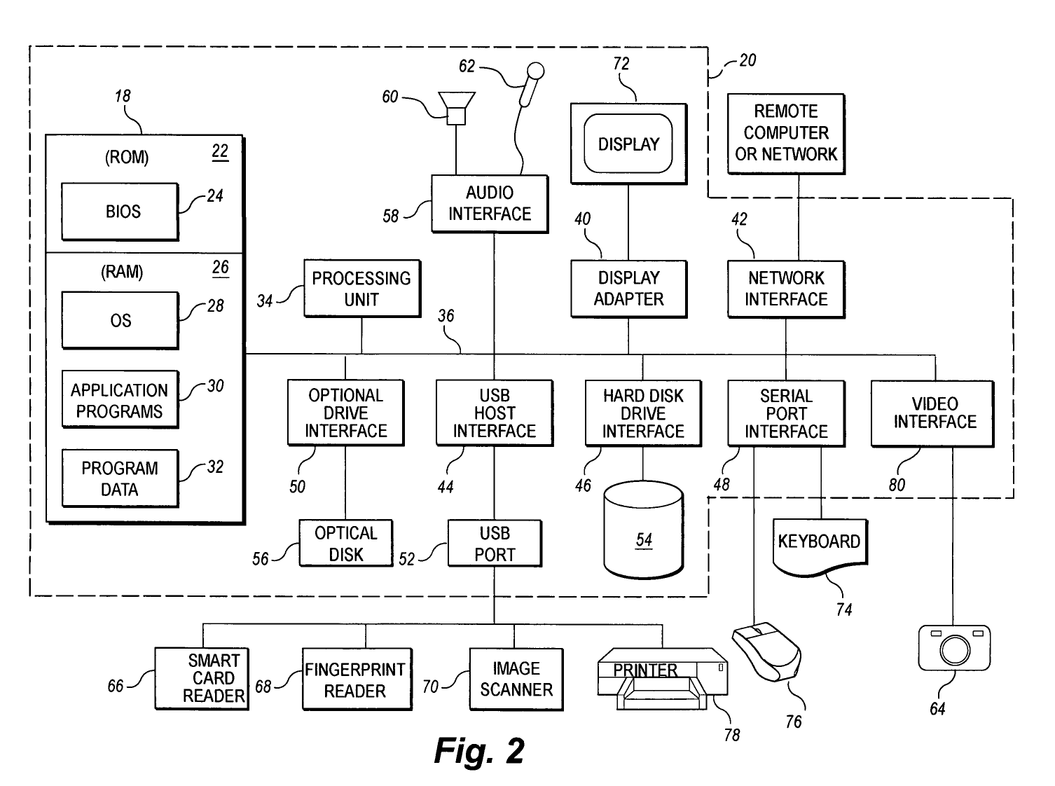

FIG. 2 illustrates an example of a computer system 20 on which the invention may be implemented. For example, one or more of such computer systems may be employed as the interview workstation 124, central station computer 104a-104c, or database server 112 described above and shown in FIG. 1. The computer system 20 includes a system bus 36 that interconnects various elements making up the computer 20. A processor subsystem 34, a system memory 18, and a number of interface and adapter systems supporting the connection of system and external devices to the computer are connected to the system bus 36.

The system bus 36 may be comprised of a number of bus subsystems. For example, a PCI bus, an ISA bus, and an AGP connection system may be provided. The system bus provides the means for moving data between the various components of the personal computer 20.

The processor subsystem 34 comprises one or more central processing units (CPU). The processor subsystem directs the overall operation of the computer by retrieving and executing logical and arithmetic instructions and issuing commands to control other computer components.

The system memory 18 may comprise read only memory (ROM) 22 and random access memory (RAM) 26 components. The ROM 22 includes the basic input/output system (BIOS) 24. The BIOS 24 includes routines that are performed to initialize the computer during startup and to conduct low-level operations between system components.

The RAM 26 may store the operating system 28, and may store instructions and data for executing application programs in the application program 30 and program data 32 areas. The information stored in the RAM 26 may be loaded from a non-volatile storage area, such as ROM 22 or a hard drive 54, during startup of the system or upon initiation of an application program.

Additional elements connected to the system bus 36 may include a display adapter 40, a network interface 42, a USB host interface 44, a hard disk drive 46 interface, a serial port interface 48, a video interface 80, and an audio interface 58.

The display adapter 40 provides an interface for connecting an external monitor or display 72 to the computer. The network interface 42 provides a network connection capability, allowing the computer to communicate to remote devices through a local area network or the Internet. The USB host interface 44 is connected to the system bus 36 and provides an interface to a USB port 52. The USB port 52 may supply electrical power and a data path for one or more USB compatible peripherals. The examples of peripheral devices that may be connected to the computer system 20 include a smart card reader 66 for reading and writing data stored on a smart card; a biometrics input device, such as a fingerprint reader 68 for scanning fingerprint data into the computer 20; an image scanner 70 for scanning images and sending image data to the computer system 20; and a printer 78 for providing a card copy of data and images stored in or generated by the computer system 20.

One or more hard drives 54 may be connected to the computer 20 through the hard disk drive interface 46. The hard drive 54 provides non-volatile storage and may store the operating system, application programs, and program data.

Input devices for accepting data input from users, such as a mouse 76, keyboard 74, or trackball (not shown), may be interfaced to the system bus 36 via the serial port interface 48. Alternatively, a USB compatible keyboard, mouse, or other input device may be connected to the computer via the USB port 52.

Additional devices, such as an optical disk drive 56 for reading from or writing to a removable optical disk, such as a CD-ROM, CD-R, DVD-ROM or other optical media, may be connected to the computer through an optical drive interface 50.

A video interface 80 may be provided to facilitate the connection of a video device, such as a video camera 64, for the purpose of supplying video data to the computer. An audio interface 58 may be provided for the purpose of receiving and converting audio data from an audio input device, such as a microphone 62 for input into the computer system, or for converting audio data from the computer into an audible output to a speaker 60.

Those skilled in the art should appreciate that the list of components given above is not an exhaustive one, and that other types of devices can be interfaced to the computer using the interfaces described above or through the use of additional interfaces and controllers connected to the system bus 36. Additionally, components and their associated interfaces may be omitted from a computer when their functionality is not required for a particular function or purpose of the computer. For example, the keyboard, mouse, and monitor may be omitted from a computer for which direct interactive input from a user is not required.

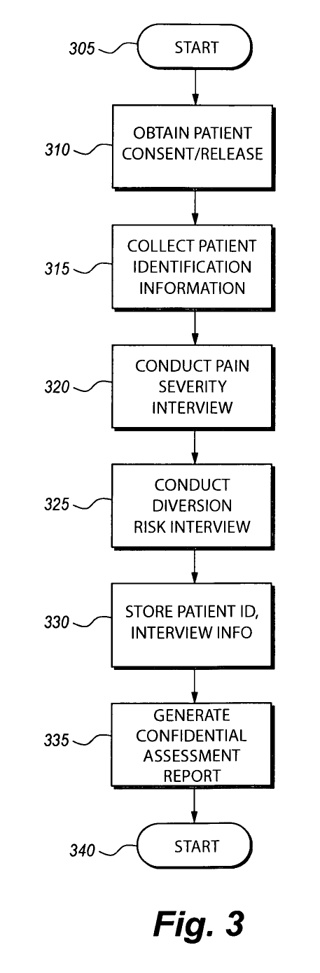

Referring now to FIG. 3, the process for assessing patient suitability for receiving controlled substance medication to treat pain is described. A patient is a person who alleges or shows physical indicia to a treating physician indicating that the person is suffering from pain, and whom the treating physician has tentatively identified as a candidate for a pain treatment strategy requiring the use of a controlled substance medication.

Once the physician has identified a patient as a candidate for a controlled substance, an assessment process verifying the patient's suitability as a candidate begins at the start block 305. At block 310, the patient's consent is obtained for beginning the process. In addition, the patient's consent may be obtained and includes documenting on a signed form the collection of medical information and permission to subject the patient to deception detection analysis for the purpose of verifying the truth of the patient's responses to the interview process. Software residing in computer readable medium accessible to a computer, such as the interview workstation (124 in FIG. 1), may direct the operations of the computer to collect the release information by scanning in the signed document on an image or document scanner attached to the computer. Alternatively, an interviewer may simply record the obtaining of the patient's consent at a user interface presented on the display monitor under control of software stored on the interview workstation. Information included with the patient's consent may additionally designate individuals authorized to receive confidential medical information associated with the patient.

After the patient's consent has been obtained, process operation proceeds to block 315 where information relating to the identity of the patient is collected. The information includes the patient's name and may additionally include identifiers, such as an identifier from a government issued identification card, a social security number, or biometric information. The identifying information may be collected under control of software residing in a computer readable medium accessible to a computer, such as the interviewing workstation (124 in FIG. 1).

The patient's name and identification may be entered in a number of ways. For example, the biometric information may be read from the patient using an appropriate input device. The patient's fingerprints may be read in from a finger print reader attached to the interview workstation. The patient's facial features may be recorded using a video or digital camera connected or sent to the computer. Photographic information, such as that present on a government issued identification card, may be scanned in and stored in computer readable medium using an image scanner attached to a computer. The patient's name, and other identifying information, such as biometric information, may be read from memory residing on a smart card using a smart card reader connected to a computer. Alternatively, a user interface may be generated and displayed on the computer monitor under the control of executing software residing in computer readable medium accessible to the computer, and identifying information may be entered via the user interface using an input device, such as a computer keyboard.

Once the required identification information has been collected from the patient, the process continues at blocks 320 and 325, which comprise processes for conducting interviews to assess the pain suffered by the patient and the risk that the patient will divert prescribed medications for uses other than the treatment of pain as prescribed by the physician. Diversion is the use of controlled substance medication for purposes other than the treatment of pain in accordance with a program legitimately prescribed by a physician. Diversion includes the taking of controlled substance medication by the patient for purposes other than treating pain as prescribed, or any use by persons other than the patient for whom the controlled substance medication was prescribed. Diversion activities include recreational use or mood altering uses by the patient; appropriation by persons, such as members of the patient's household, with or without the consent of the patient; and sale or distribution of the control substance medication by the patient. Diversion risk is the qualitative or quantitative probability of the occurrence of diversion activities.

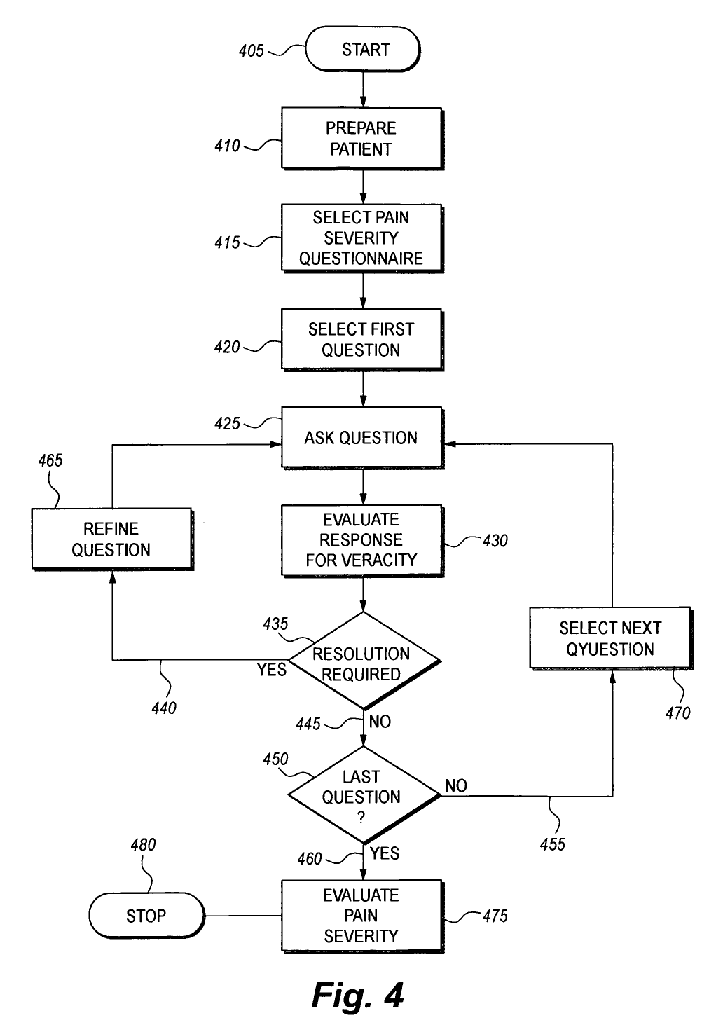

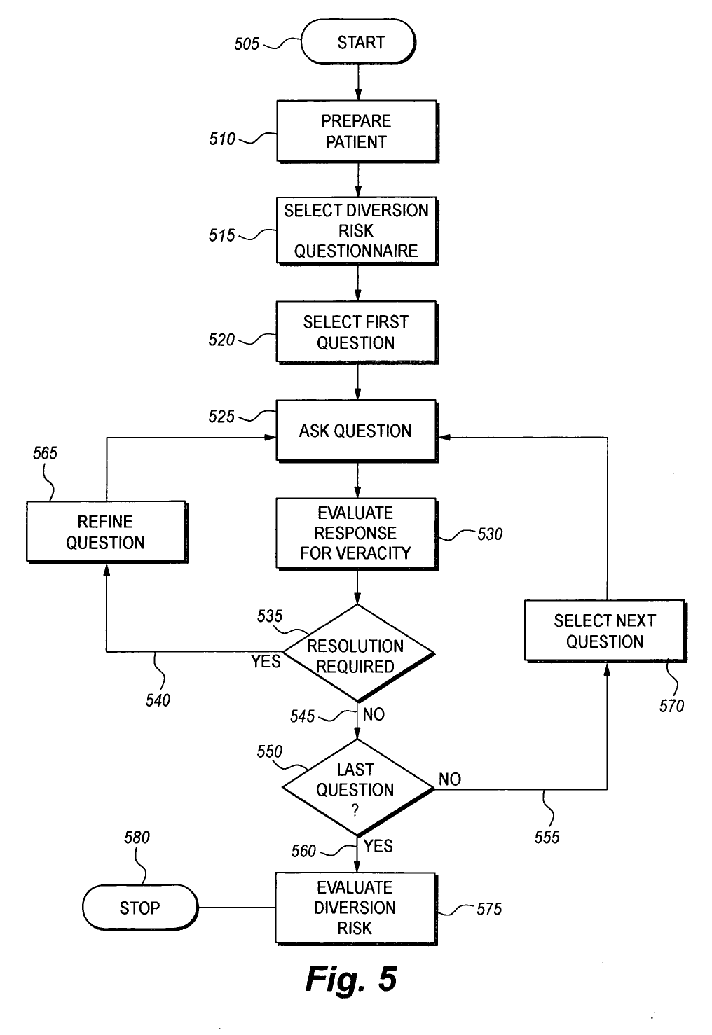

While the pain and diversion risk assessments are represented as separate sequentially conducted interviews in blocks 320 and 325 of FIG. 3, the interviews may be combined into a single interview. When conducted separately, the pain assessment and risk diversion interviews may be conducted in any order. Details of the processes for assessing a patient's pain and assessing the risk that the patient will divert prescribed drugs will be described by referring to FIGS. 4 and 5.

As depicted in FIG. 4, the process for assessing the pain suffered by the patient begins at block 405 labeled “Start”. In the first step of the process represented by block 410, the patient is prepared for the pain assessment interview process. As will be later described herein, the process employs psycho/physiological deception detection surveillance (PDDS) tools as a means of detecting attempts by the patient to provide deceptive interview response(s) for the purpose of obtaining controlled substance medications. Because PDDS tools measure the stress generated when a person attempts to vocalize a deceptive statement, the patient preparation step 410 is designed to establish that the patient has the required investment in the process so that the patient will feel stress when providing a false response. Some PDDS tools additionally require a calibration process under which the stress responses of a person are measured while making statements known to be false or known to be true. The pain preparation step includes any calibration and pre-stress steps associated with the particular PDDS tool to be employed.

In block 415, the pain severity questionnaire is selected. The pain severity questionnaire is the set of interview questions that will be asked of the patient. The questions are designed to probe the severity and frequency of pain suffered by the patient. In one embodiment, the questions identify patient activities during different periods of a day and ask the patient to quantify the pain experience during those activities. In selecting the questions making up the questionnaire to be used with a particular patient, such factors as the patient's past interview history and truthfulness may be considered. Information concerning the patient's interview history may be stored in data structures in computer readable medium, such as storage space in the centralized database server (112 in FIG. 1). Software instructions stored in a computer readable medium contained in a computer, such as the interview workstation (124 in FIG. 1), may direct the retrieval of patient history containing data structures from the central database server under the direction of the retrieved data structures and software instructions. The interview workstation computer can select an appropriate questionnaire from a set of stored question lists, or select a sequence of questions from a stored set of questions. For example, if the retrieved interview history indicates a previous pattern of verified pain and suffering, questions comparing previous pain levels to current ones may be selected.

Once a questionnaire is selected, the process continues at block 420, and a first question from the questionnaire is selected. The selected question is then posed to the patient being interviewed at block 425, and a response is elicited from the patient. In one embodiment, the patient is prompted to respond orally, as is consistent with the application of PDDS tools to evaluate the patient's response. The patient may be prompted to respond by having the selected question read aloud by a human interviewer. Alternatively, the speech generation software in a computer readable medium may convert the stored text of a question to speech, and provide an audio rendition of the question via a speaker interfaced to an audio interface of the computer.

Once the selected question has been posed to the patient, the patient's oral response is evaluated for veracity at block 430. In a preferred embodiment, the truthfulness of the patient's response is evaluated by using a PDDS tool.

PDDS tools attempt to detect biological indicators of the stress generated when a person attempts a deceptive response. The employed PDDS tool may be chosen from such tools as polygraphs; voice stress analysis tools, such as Layered Voice Analysis (LVA) or Computer Voice Stress Analysis (CVSA); facial gesture analysis tools, such as Silent Talker; brain activity monitoring tools; such as “brain fingerprinting”; or any other tool for monitoring patient stress while providing an oral response.

If the invention uses a polygraph, sensors are connected to the patient to measure one or more biological indicators of stress, including respiration rate, skin conductivity, and cardiovascular reaction, such as systolic blood pressure. During the patient preparation step 410 described above, the polygraph sensors are connected to the patient, and the patient's stress pattern is established by correlating measured changes in biological parameters when the patient makes statements known to be true and statements known to be false. The biological indicators may be recorded on a display associated with the polygraph. Alternatively, software instructions stored on a computer readable medium in the interview workstation computer may direct the computer to collect biological data from a polygraph connected to the computer via an interface, such as a USB port. An operator may correlate the patient's polygraph responses to posed questions via a user interface displayed on the interview workstation during patient preparation (block 410) or interview response evaluation (block 430).

If the invention uses voice stress analysis, the patient's responses are collected using a microphone. For example, when CVSA is used, under the direction of software stored in a computer readable medium, such as computer readable memory contained in the interview workstation computer, the patient's response to a posed question is collected and stored using a microphone connected to a computer via an audio interface. Software residing on the computer analyses the response to detect stress patterns in the voice of the patient that indicate attempts by the patient to give a deceptive response to a question. The correlation of the response and deception indication may be conducted automatically by the computer, or, alternatively, the correlation may be provided by a human operator via a user interface displayed on the monitor of the computer.

If the invention uses facial gesture analysis as the PDDS tool, a procedure analogous to that using CVSA may be employed. For example, a facial gesture analysis tool, such as Silent Talker, may comprise software instructions stored in a computer readable medium, such as memory contained in the interview workstation computer, which direct the acquisition of video information captured using a video camera directed at the face of the patient and connected to a computer via an video interface. Software residing on the computer analyses the captured video information and detects changes in the facial features of the patient that indicate attempts by the patient to give a deceptive response to a question. The correlation of the response and deception indication information extracted from the captured video information may be conducted automatically by the computer, or, alternatively, the correlation may be provided by using input from a human operator via a user interface displayed on the monitor of the computer.

If the invention uses brain activity analysis as the PDDS tool, a procedure analogous to that using CVSA may be employed. For example, a brain-fingerprinting tool may employ sensors attached to a patient for detecting electrical activity of the patient's brain. The sensors may be connected to a computer using a suitable interface, and the output from the sensors may be captured under the control of software instructions stored in a computer readable medium, such as computer RAM. Software residing on the computer analyses the captured brain activity information and detects data patterns indicating attempts by the patient to give a deceptive response to a question. The correlation of the response and deception indication information extracted from the captured video information may be conducted automatically by the computer, or, alternatively, the correlation may be provided by using input from a human operator via a user interface displayed on the monitor of the computer.

The outcome of response evaluation step 430 is an indication that the response of the patient involved an attempt at deception, that no deception was attempted, or that the evaluation was inconclusive with respect to whether the patient attempted to deceive. After the response has been evaluated and the evaluation correlated with the posed question, the process continues with block 435.

Once the patient's response and the indication of truthfulness have been correlated with a posed question, the response is analyzed to determine the need to probe more deeply into a specific area of inquiry at block 435. For example, at step 425, the patient may be asked to specify the patient's sleeping hours. If the patient's response indicates that the patient has a small sleep period, then it may be desired to pose questions probing a difficult in achieving sleep. In another example, a deception attempt detected at block 430 may have an innocent explanation. A patient asked a question about a level of pain experienced during a certain activity might produce a stressed reaction because the level of pain may vary on different occasions. Questions probing the subject area generating the stress in more detail may be selected to resolve an indication of deception. If the deception detection result at block 430 is inconclusive, it may be desirable to repeat the question, possibly with different phrasing.

If, at block 435, it is determined that further resolution is necessary, the process continues along path 440 to block 465 for obtaining more detail concerning a specific subject area of inquiry. At block 465, refining questions are selected, and the refining question is posed to the patient at block 425. If, at block 435, it is determined that further resolution is not necessary, the process continues along path 445 to block 450.

At block 450, it is determined whether the last question of the questionnaire has been asked. When all of the selected questions and any refining questions have been asked and responded to, then the pain severity interview is complete, and the process continues along path 460 to block 475. If additional interview questions remain to be asked, the process continues along path 455 to block 470 where the next question in the questionnaire is selected and then posed to the patient at block 425.

At block 475, the stored correlation responses of the patient and the indications of deception with each posed interview question are analyzed to generate information related to the pain suffered by the patient. In analyzing the patient's pain, deceptive responses may be discounted completely using a Boolean logic based analysis, or, alternatively, may be discounted in a weighted fashion using a fuzzy logic analysis in which the probability of deception is provided as an outcome of the PDDS analysis. When a refining response is obtained, the refining responses may be substituted for an initially posed question when the analysis suggests that the follow on refining responses are more accurate.

Alternatively, the refining responses may be discounted using fuzzy logic based on the indication of truthfulness and the nature of the response to the refining questions. As a result of the analysis performed at block 475, an overall pain severity level experienced by the patient may be computed. The pain level experience during specific activities may be identified, and a reliability index based on the patient's deceptive and non-deceptive responses may be computed. The pain severity analysis may be directed by software instruction stored in a computer readable medium and executed on a computer, such as the interview workstation (124 in FIG. 1). The results of the pain severity interview may be stored in a computer readable medium.

Once the pain severity assessment is complete, the pain severity assessment process terminates at block 480.

Referring now to FIG. 5, the process for interviewing a patient to determine the risk of diversion of controlled substances for an illicit purpose is described. The diversion risk interview process described with reference to FIG. 5 is highly analogous to the pain severity risk process described above with reference to FIG. 4.

As depicted in FIG. 5, the process for assessing the diversion risk associated with a patient is entered at block 505 labeled “Start”. Upon entering the process, the process execution begins at block 510, in which the patient is prepared for the diversion risk interview process. The patient preparation step 510 provides the preparation necessary for the use of PDDS deception detection tools and is essentially identical to the step 410 described above with reference to FIG. 4. In one embodiment, the pain severity and risk diversion interviews are either combined or performed sequentially, and the patient preparatory steps 410 and 510 are combined and performed once per interview session. Alternatively, it may be necessary to provide a separate patient preparatory step if the pain assessment interviews and the risk diversion interviews are separated in time rather than being conducted concurrently or sequentially. As has been described above, the interview process employs psycho/physiological deception detection surveillance (PDDS) tools as a means of detecting attempts by the patient to provide deceptive interview response(s) for the purpose of obtaining controlled substance medications.

The diversion risk assessment process continues at block 515, at which the diversion risk questionnaire is selected. The diversion questionnaire is the set of interview questions that will be asked of the patient. The questions are designed to probe the factors indicating that pain medication prescribed for treatment of a patient's pain will be used for other purposes, such as recreational use by the patient, as a mood-altering substance, or used by persons other than the patient. In a preferred embodiment, the questions identify behavior patterns of the patient and of persons residing or associated with the patient that indicate a risk that the patient will misuse prescribed controlled substances. In selecting the questions making up the questionnaire to be used with a particular patient, such factors as the patient's past interview history and truthfulness may be considered. Background information indicating a history of misuse of controlled substances may also be included. Information concerning the patient's interview history may be stored in data structures in a computer readable medium, such as in the centralized database server (112 in FIG. 1). Software instructions stored in a computer readable medium contained in a computer, such as the interview workstation (124 in FIG. 1), may direct the retrieval of patient history containing data structures from the central database server. Under the direction of the retrieved data structures and software instructions, the interview workstation computer selects an appropriate questionnaire from a set of stored question lists, or selects a sequence of questions from a stored set of questions.

For example, if the retrieved interview history indicates past diversion, drug abuse, or fraudulent means to obtain prescriptions to controlled substances, a questionnaire containing questions intended to probe these areas may be selected or generated. Information related to the background of a patient may be retrieved from an external database, such as database servers 118 and 120, as shown in FIG. 1. The external servers may contain information related to past drug/controlled substances related convictions that may be used to identify areas requiring detailed probing to assess a current risk of diversion. Background information generated from surveillance activities conducted by an operator of the inventive system may be stored and retrieved from the centralized database server 112 of FIG. 1. For example, information from pill counts, drug testing results, or surveillance conducted by or at the direction of the system operator, may be stored in data structures residing on the centralized database server 1112.

Once a questionnaire is selected, the process continues at block 520, and a first question from the questionnaire is selected. The selected question is then posed to the patient being interviewed at block 525, and a response is elicited from the patient. In a preferred embodiment, the patient is prompted to respond orally, as is consistent with the application of PDDS tools to evaluate the patient's response. The posing of questions to the patient and prompting for a response is performed in a matter identical to that described at block 425 in FIG. 4, described above.

Once the selected question has been posed to the patient, the patient's oral response is collected and evaluated for veracity at block 530. In a preferred embodiment, the truthfulness of the patient's response is evaluated by using a PDDS tool. The collection of the patient's response, the operation of the PDDS tool for evaluating the response of a patient, and the correlation of the response and the deception indication with the posed question, are all identical to that described for the pain severity interview above.

For questions that are selected to probe background information collected by the operator or collected from an external database, the truthfulness evaluation may additionally include a comparison of the patient's response to information in the patient's background data.

The outcome of response evaluation step 530 is an indication that the response of the patient involved an attempt at deception, that no deception was attempted, or that the evaluation was inconclusive with respect to whether the patient attempted to deceive. After the response has been evaluated and the evaluation correlated with the posed question, the process continues with block 535.

Once the patient's response and the indication of truthfulness have been correlated with a posed question, the response is analyzed to determine the need to probe more deeply into a specific area of inquiry at block 535. For example, at step 525 the patient may be asked whether the patient has ever provided pain medication prescribed for the patient to another person. If the patient's response indicates that the patient has provided medication to another person, questions probing this activity in more detail may be selected or generated in order to clarify the circumstance under which this activity occurred. Questions involving the nature and type of the medication involved may be asked. In another example, a deception attempt detected at block 530 may have an innocent explanation. Stress generated when a patient is asked a question about providing a controlled substance medication to another person may be generated because the patient is unsure whether a prescribed medication, such as ibuprofen, constitutes a controlled substance. Questions probing this area in more detail may be selected to resolve the indication of deception. If the deception detection result at block 530 is inconclusive, it may be desirable to repeat the posed question possibly with different phrasing.

If, at block 535, it is determined that further resolution is necessary, the process continues along path 540 to block 565. At block 565, refining questions are selected, and the refining question is posed to the patient at block 525. If, at block 535, it is determined that further resolution is not necessary, the process continues along path 545 to block 550.

At block 550 it is determined whether the last question of the questionnaire has been asked. When all of the selected questions and any refining questions have been asked and responded to, then the diversion risk interview is complete, and the process continues along path 560 to block 575. If additional interview questions remain to be asked, the process continues along path 555 to block 570 where the next question in the questionnaire is selected and then posed to the patient at block 525.

At block 575, the stored correlation responses of the patient and the indications of deception with each posed interview question are analyzed to generate information related to the risk of diversion of a prescribed controlled substance medication by the patient. In analyzing the diversion risk, deceptive responses may be discounted completely using a Boolean logic based analysis, or, alternatively, may be discounted in a weighted fashion using a fuzzy logic measurement in which the probability of deception is obtained as a result of the PDDS analysis. When a refining response is obtained, the refining responses may be substituted for an initially posed question when the analysis suggests that the refining responses are more accurate.

Alternatively, the refining responses may be discounted using fuzzy logic based on the indication of truthfulness, the strength of indication of a given question, and the nature of the response to the refining questions. When truth assessments are made based on comparison with external data, a reliability index associated with the external data may be used as a weighing factor to discount the assessments made using that data. The reliability index may be correlated with the source of the data, or may be associated with a specific item of external data. As a result of the analysis performed at block 575, an overall diversion risk associated with the interviewed patient may be computed.

A specific diversion risk, such as a risk of personal misuse, or of selling controlled substances to others, or of having persons in the patient's residence misappropriate controlled substance medications without the patient's consent, may be identified, and a reliability index based on the patient's deceptive and non-deceptive responses may be computed. The diversion risk analysis may be directed by software instruction stored in a computer readable medium and executed on a computer, such as the interview workstation (124 in FIG. 1). The results of the diversion interview may be stored in a computer readable medium.

The assessment of diversion risk may also include detection of repeated attempts by a patient to obtain prescriptions for controlled substance medications within an unreasonably short period. A complicating factor in detecting repeated attempts is that the patient may attempt to disguise his identity by giving a false name during an interview session. Identifying data obtained from the patient during the assessment process may be used to query the database for matches to detect disguised attempts. For example, biometric data such as fingerprints, or photographic data of the patient's features or identifiers from a government issued identification card obtained during an interview, may be used to identify interview records in the database belonging to a patient. Software residing on a system computer may comprise image-matching routines, fingerprint matching routines, or other sets of executable instructions appropriate for the given set of identifying information. By using biometric data, such as fingerprints, photographs, or other identifying data that are difficult to falsify, attempts by patients to disguise their identity may be detected or deterred.

Once the diversion risk assessment is complete, the diversion risk assessment process terminates at block 580.

Returning now to FIG. 3, it may be appreciated that after the interviewing processes of blocks 320 and 325 are completed, the process for assessing patient suitability for receiving controlled substance medication continues at block 330 where the interview results are correlated with the patent identification information and stored in a computer readable medium for later retrieval. Preferably the results are stored on a centrally accessible server, such as the central database server 112 in FIG. 1.

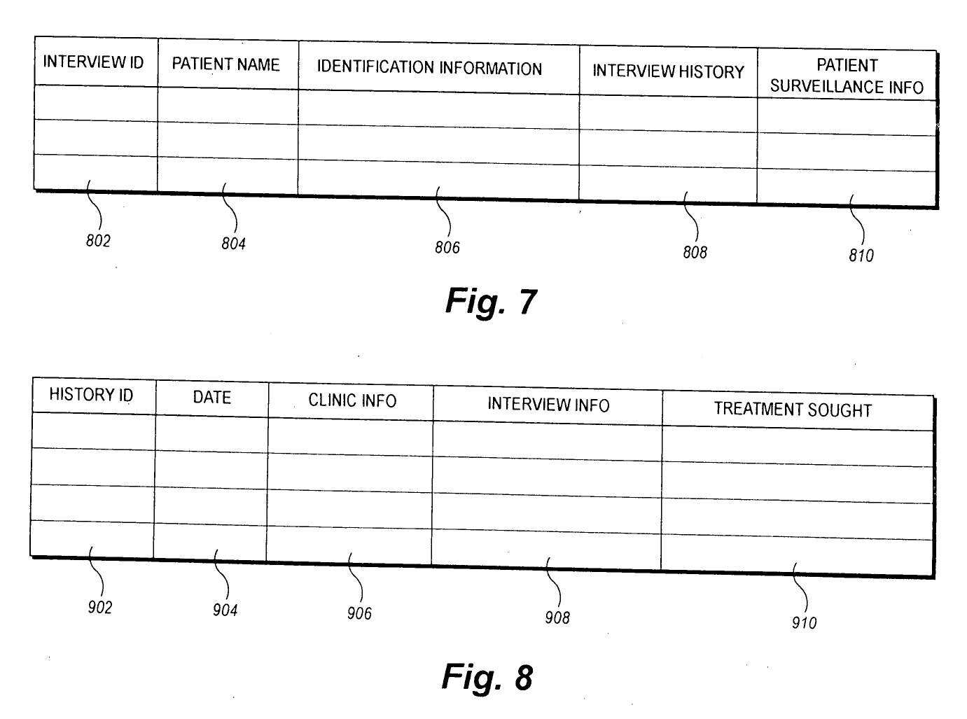

Data structures for storing the correlated information in accordance with an embodiment of the invention will be described with reference to FIGS. 7 and 8.

Referring first to FIG. 7, a patient information table is described. The patient information table comprises one or more data records holding data specific to an interview session conducted for a patient. The interview data is organized into fields holding one or more data items associated with an interview session. The first data field shown is the Interview ID field 802. The interview ID field 802 contains an identifier unique to a record in the interview information table. The interview ID identifier 802 may be a sequentially generated numeric identifier generated from a centralized source.

Alternatively, the identifier may be a computed identifier, such as one combined with a computed hashing of the stored contents of a record in the database. Any method known in the art that guarantees the uniqueness of the identifier may be used. The patient name field 804 contains the name of the interviewed patient. The identification information field 806 comprises information collected to identify the patient. As described above, the patient identification field 806 may contain digitized identifying data, such as biometric data including fingerprints, government issued identification information, photographic information, or description of unique physical characteristics of the patient.

Records in the interview information table also contain an interview history field 808. The interview history field 808 contains data describing an interview conducted in accordance with the invention. The interview history field 808 may directly contain the interview information, or, alternatively, may contain an index usable to retrieve interview information from a data structure in a separate table, such as that shown in FIG. 8 and described below. Each data record in the patient interview table may further comprise a patient surveillance info field 810. The patient surveillance info field contains information collected by a system operator relevant to the patient. For example, the patient surveillance info field 810 may contain drug-testing results, such as urinalysis, pill counts to monitor medication usage, informer reports, and other information relevant to possible misuse of controlled substance medication by the patient.

Referring now to FIG. 8, a second data structure comprising a database table will be described. The data structure is an interview history table containing the details of a completed interview session. The interview history table comprises one or more data records, each holding the data for one interview session. Each record contains one or more information fields holding a specific set of information concerning the interview session. A first field of a record in the interview history table is the history identification or “History ID” field 902. The History ID field provides a unique index identifying a specific record in the interview history table. The index may be used as the interview history field 808 of the patient information table to correlate an identified patient interview record with the details of a specific interview history table record.

The record in the interview history table further comprises a date field 904 containing the date of the interview session. By querying a database for interview dates for a given patient, a patient's pattern of interviews can be detected. The pattern of frequent interviews to obtain controlled substance medications may indicate that the patient is seeking to obtain the medications for an illicit purpose. Additionally, data fields in the interview history records allow a more refined evaluation of the patient history. The record may include a clinic info field 906 containing data identifying the clinic or doctor from which the patient is seeking treatment.

Queries retrieving the clinic info field 906 allow identifying attempts by the patient to disguise his attempts to obtain medication by visiting multiple prescribing physicians over a short period of time. Queries retrieving the clinic info field 906 would also allow identifying abnormal prescription patterns involving a specific doctor or clinic. Each interview history record may further comprise an interview info field 908 containing the results of the diversion risk and pain severity interviews conducted during a particular interview session. The interview info field may further contain release information identifying parties whom the patient authorizes to access the patient's medical information. Queries retrieving the interview info field 908 can be used to establish a patient's track record of deception, or reliable and proper use of prescribed controlled substance medication. Additionally, the field may be used to develop the patient's pain suffering patterns. The database records may additionally comprise a Treatment Sought field 910 identifying the medication targeted for the patient.

Referring again to FIG. 1, it may be appreciated that centralized database server 112 is accessible to computers attached to the central network 110, such as the central station computers 104a-104c or the interview workstation 124. Access to the database server may require authentication. Authenticated access to a patient's information may be controlled by patient release information stored in data structures stored in the central computer database. Queries to retrieve the data structures from the centralized database server can be generated under the control of software instructions stored in computer readable medium on computers, such as the central station computers 104a-104c or the interview workstation 124. As described above, the retrieved data structures control the operation of the system to generate or select the pain severity and risk diversion questionnaire, and direct application of PDDS tools to detect patient deception and to control the flow of patient interview sessions, among other described functionality.

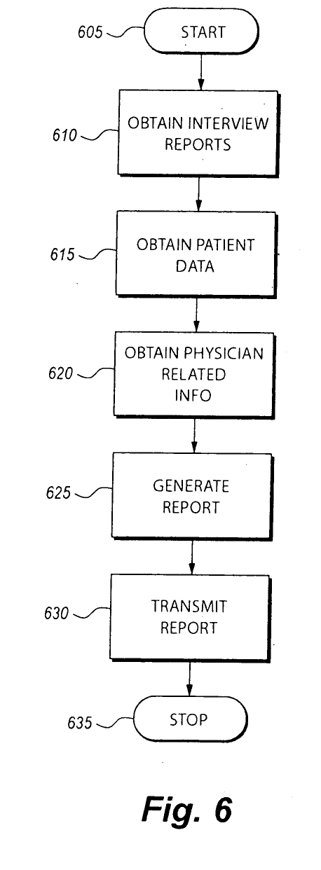

Returning to FIG. 3, it may be appreciated that the method for assessing the suitability of a patient to receive a controlled substance medication further comprises generating a confidential assessment report at block 335. After generating the report, the process terminates at the “Stop” 340. Details of the process for generating the confidential assessment report are described with reference to FIG. 6.

The process illustrated in FIG. 6 may be carried out on a computer connected to the central network 110 in FIG. 1, such as a central station computer 104a executing instructions stored in a computer readable medium accessible to the computer. The report generating process is entered at starting block 605, and execution of the process begins at blocks 610, 615, and 620 with the retrieval of information required to generate the contents of the confidential assessment report. Queries generated on a central workstation computer retrieve the data structures, described above with reference to FIGS. 7 and 8, containing information on one or more interview sessions conducted for the patient. Data in the retrieved data structures may be parsed to retrieve the interview reports (block 610), patient identifying information, background information, and interview history (block 615) and physician identifying information (block 620). At block 615, the retrieval of patient related information may further comprise querying external databases in remote database servers, such as servers 118 or 120 in FIG. 1, using the patient identifying information to retrieve relevant information concerning the patient. For example, the patient's criminal history may be retrieved and reviewed for the occurrence of drug-related offenses relevant to a risk of diversion of controlled substances by the patient.

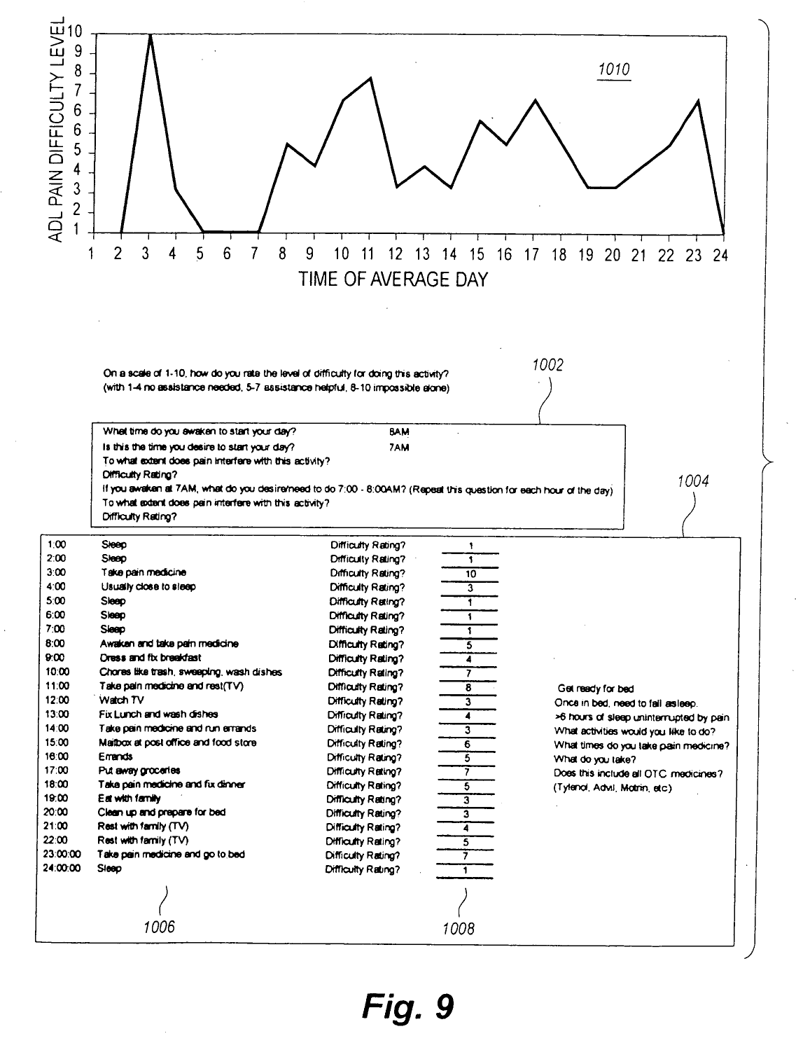

Following the retrieval of information, the process continues at block 625, at which the confidential assessment report is generated. The report comprises a summary of the relevant information associated with the patient's background and interview information retrieved, as described above. In one embodiment, the report includes summarized assessments of the pain suffered by the patient and of the risk of diversion of controlled substances associated with the patient. For example, based on the computed overall pain severity level, the patient's pain severity may be described as mild or severe. The pain description may be amplified by a description of the pain suffered by the patient. The pain severity summary may further include a comparison to past levels of pain suffered by the patient. The pain severity summary may include a complete description of the questions posed to the patient and the patient's responses may be provided, allowing a physician to make an independent assessment of the patient's pain severity. An embodiment of a patient pain survey response report is described with reference to FIG. 9.

The pain severity patient response report comprises an initial question section 1002 containing an initial question posed to the patient. The patient is queried for the patients sleeping time period, and the patient's difficulty with sleeping. Based on the response to this question the patient is then asked follow-on questions concerning his sleeping and his activities when awake for each hour of the day. The patient's activities and reported pain severity levels are reported in the detailed summary section 1004. The detailed summary section contains a list of each activity 1006 and a difficulty level caused by the patient's pain 1008 for each hour of the day. The pain severity patient response report may further comprise a graphical display 1010 of the quantified pain level for the patient for each hour of the day.

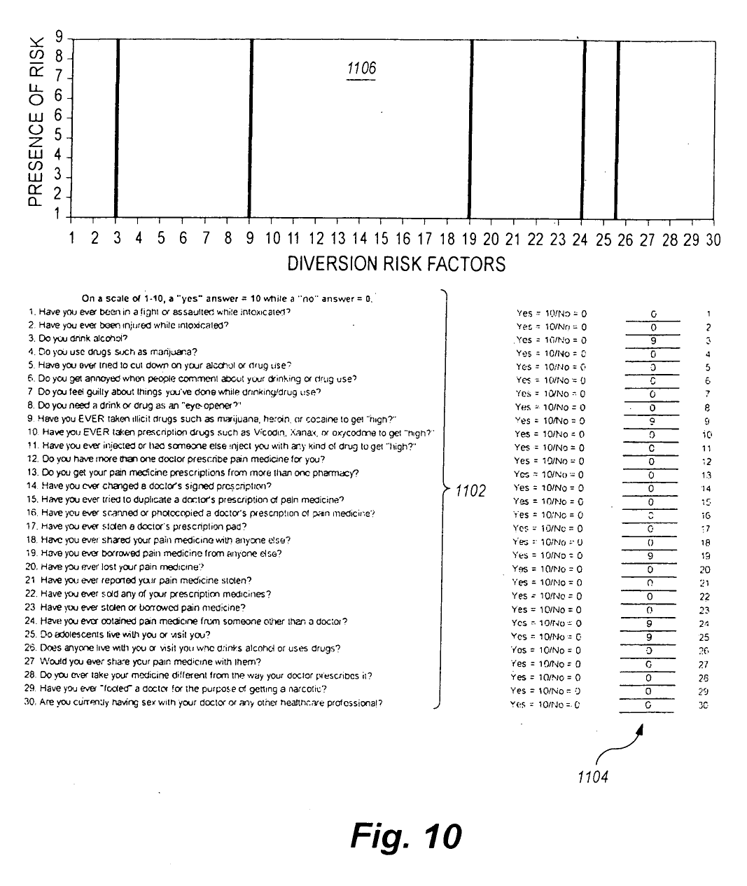

The diversion risk assessment of the confidential assessment report may comprise a characterization of the risk of diversion of controlled substance medication by the patient based on the computed overall diversion risk described above. For example, the risk may be characterized as low, moderate, or high. The diversion risk assessment may further comprise a description of the particular items contributing to the calculated diversion risk. For example, the patient's response to questions that indicate a risk of diversion may be specifically identified, items in the patient's background indicating a history of diverting controlled substances may be described, and attempts at deception by the patient identified during an interview may be reported. The diversion risk assessment may include all relevant information identified during the interview, background checks, or past surveillance of the patient, allowing a physician to make an independent assessment of the risk of diversion associated with a patient. An embodiment of a diversion risk patient survey response report is described with reference to FIG. 10.

The diversion patient response report comprises a question section listing each of the questions posed to the patient 1102 and a patient response listing 1104 correlating the response of a patient to each question posed by the interviewer to the patient. The pain severity patient response report may further comprise a graphical display 1106 in which highlighted bars are used to represent each patient response that suggests a risk that the patient might attempt to divert controlled substance medications for illicit use.

Returning to FIG. 6, it may be appreciated that after the patient assessment report is generated at step 625, the process continues at step 630, at which the report is transmitted for receipt by authorized personnel. In one embodiment, the report is transmitted in accordance with the consent and release forms executed by the patient. Transmission of the report may comprise retrieving the report from a computer readable medium, such as by retrieving data structures stored on the central server (112 in FIG. 1). A hard copy report may be generated by printing the generated report on a printer attached to a computer, such as a central workstation computer (such as 104a in FIG. 1), or on a portable computer connected directly or indirectly to the central network (110 in FIG. 1). Alternatively, the report may be stored in a secure storage area on a central workstation computer with networked access to the computer controlled via data structures containing information specifying personnel authorized to access the patient's assessment report. As may be appreciated by one of skill in the art, alternate methods of transferring the report, such as email or transmission over a network to the recipient, may be used to transmit the report.

After the report is transmitted in block 630, the process for generating the patient assessment report terminates at the “Stop” block 635.

The term “computer readable medium” as used herein refers to any medium that participates in providing instructions to a computer processor for execution. Such a medium may take many forms, including but not limited to, non-volatile media, volatile media, and transmission media. Non-volatile media includes, for example, optical or magnetic disks. Volatile media includes dynamic memory, such as main memory. Transmission media includes coaxial cables, copper wire and fiber optics, including the wires that comprise a computer bus. Transmission media can also take the form of acoustic or electromagnetic waves, such as those generated during facsimile, radio wave and infrared data communications.

In the above description, the term database is generally used to refer to one or more data records or a collection of data regarding various types of information or data. References to specific databases described herein are not intended to require a particular structure or organization of physical or logical databases. As would be apparent to one of ordinary skill in the art after reading this document, varying physical or logical data groupings can be provided in one or more locations or on one or more devices to implement the described databases.

It is to be understood that the present invention is not limited to the embodiments described above, but encompasses any and all embodiments within the scope of the following claims.

We claim:

1. A psycho/physiological deception detection method for assessing patients, comprising the steps of:
obtaining identifying information from the patient;
conducting a patient interview, including:
selecting a plurality of questions adapted to assess the patient's suitability for receiving controlled dangerous substance medications;
for each question in the plurality of questions, posing the question to the patient, obtaining an oral response from the patient to the question, monitoring the patient using a psycho/physiological deception detection (PDDS) tool while obtaining the oral response, and assigning a deception index based on monitoring the patient using the PDDS tool; and
for each question in the plurality of questions, associating the obtained patient response and the assigned deception index with the question.

2. The psycho/physiological deception detection method according to claim 1, wherein said step of obtaining identifying information comprises obtaining an image of the patient.

3. The psycho/physiological deception detection method according to claim 1, wherein said step of obtaining identifying information comprises obtaining the fingerprints of the patient.

4. The psycho/physiological deception detection method according to claim 1, wherein the plurality of questions includes at least one question adapted to assess the risk that the patient will divert a controlled substance.

5. The psycho/physiological deception detection method according to claim 1, wherein the plurality of questions includes at least one question adapted to assess pain suffered by the patient.

6. The psycho/physiological deception detection method according to claim 1, wherein said step of monitoring the patient comprises administering a polygraph examination to the patient while obtaining the oral response.

7. The psycho/physiological deception detection method according to claim 1, wherein said step of monitoring the patient comprises conducting voice stress analysis of the patient's voice in making the oral response.

8. The psycho/physiological deception detection method according to claim 1, wherein said step of monitoring the patient comprises administering a facial gesture analysis on the patient's facial gestures when making the oral response.

9. The psycho/physiological deception detection method according to claim 1, wherein said step of monitoring the patient comprises monitoring the patient's brain activity.

10. The psycho/physiological deception detection method according to claim 1, wherein the step of assigning a deception index is performed simultaneously with the step of obtaining the oral response.

11. The psycho/physiological deception detection method according to claim 1, wherein the step of assigning a deception index is performed off-site subsequent to the interview.

12. The psycho/physiological deception detection method according to claim 1, further comprising the step of determining if refining questions are required based on the deception index for each question in the plurality of questions.

13. A computerized method for assessing a patient's suitability for receiving controlled dangerous substance medications, the method comprising the steps of:
obtaining identifying information from the patient;
checking a networked central database for a history of the patient's controlled dangerous substance medication background using the identifying information;
conducting a patient interview by posing a plurality of questions to the patient and collecting data during the interview;
monitoring the patient using a psycho/physiological deception detection (PDDS) tool during the interview;
for each posed question, assigning a deception index based on monitoring the patient using the PDDS tool and associating the deception index with the posed question;
computer-generating a report assessing pain suffered by the patient and assessing risk of personal misuse and diversion of prescribed controlled dangerous substances;
recording the report in the central database;
prescribing a controlled dangerous substance for the patient when the report shows the patient is a suitable candidate for receiving the controlled dangerous substance; and
refraining from prescribing the controlled dangerous substance when the report shows the patient presents a substantial risk for personal misuse or diversion of the controlled dangerous substance.

14. The method according to claim 13, wherein said step of checking a networked central database comprises accessing the central database over a network selected from the group consisting of an intranet, an extranet, and the Internet.

15. The method according to claim 14, further comprising the step of checking the patient's criminal record by accessing an external database.

16. The method according to claim 13, wherein the PDDS tool is selected from the group consisting of: a polygraph examination; a voice stress analysis; a facial feature analysis; and monitoring brain activity.

17. A system for assessing a patient's suitability for receiving controlled dangerous substance medications, the system comprising:
a computer workstation connected to a network;
a central database connected to the network, the central database having stored thereon a plurality of patient records searchable by patient identifying information, each of the records including the patient's controlled dangerous substance background and prior assessments of suitability for controlled dangerous substance administration using a psycho/physiological deception detection (PDDS) tool;
a biometric identification input device connected to the computer workstation for inputting biometric identification of the patient;
a PDDS tool for assessing complaints of pain by the patient;
computer software stored on the computer workstation and operable on the computer workstation, the software having:
means for checking the central database for the patient's controlled dangerous substance background and the prior assessments of suitability for controlled dangerous substance administration using the input biometric identification of the patient;
means for recording the patient's responses to questions assessing the patient's complaints of pain and a rating of the PDDS tool evaluation of each of the responses; and
means for automatically generating a report summarizing the patient's controlled dangerous substance background and prior assessments of suitability for controlled dangerous substance administration, and assessing the patient's complaint of pain and risk of controlled dangerous substance misuse or diversion resulting from the PDDS evaluation.

18. The system according to claim 17, further comprising means for transmitting the report to the central database for recording thereon.

19. The system according to claim 17, wherein the biometric identification input device comprises a device selected from the group consisting of a video camera; and a fingerprint scanner.

20. The system according to claim 17 wherein the PDDS tool is selected from the group consisting of a polygraph, a voice stress analysis machine, a facial feature analysis machine, and a brain activity monitoring machine.
